(19) United States
(12) Patent Application Publication
(10) Pub. No.: US 2004/0209812 A1
(43) Pub. Date: Oct. 21, 2004

(54) Use of erythropoietin in stroke recovery

(76) Inventors: Michael J. Renzi, Pipersville, PA (US); Michael Gold, Newton, PA (US); Kenneth James Rhodes, Hillsborough, NJ (US); Navneeth Thirumalai, Kendall Park, NJ (US); Francis Farrell, Doylestown, PA (US); Linda Jolliffe, Belle Mead, NJ (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/810,755

(22) Filed: Mar. 26, 2004

Related U.S. Application Data
(60) Provisional application No. 60/458,193, filed on Mar. 27, 2003.
(60) Provisional application No. 60/477,494, filed on Jun. 11, 2003.

(51) Int. Cl. A61K 38/18

(52) U.S. Cl. 514/12

(57) ABSTRACT

The present invention demonstrates EPO's efficacy in promoting patient recovery following an ischemic event. The invention demonstrates that a longer window of opportunity exists for the initial uses of EPO in treating ischemic stroke than had been previously been understood.

### CROSS REFERENCE TO RELATED APPLICATION(S)

This application claims the priority of provisional applications U.S. Ser. No. 60/458,193, filed Mar. 27, 2003, and U.S. Ser. No. 60/477,494, filed Jun. 11, 2003 both entitled “Use of Erythropoietin in Stroke”, the contents of which is hereby incorporated by reference in its entirety.

### TECHNICAL FIELD

The present invention provides different dosing erythropoietin (EPO) dosing regimens to promote recovery after an ischemic event such as stroke.

### BACKGROUND

The term stroke refers to an abrupt impairment of brain function resulting from the occlusion or rupture of an intra- or extracranial blood vessel. It occurs when one or more blood vessels in or leading to the brain ruptures or are clogged by a thrombus, atherosclerotic plaque or some other particle(s). As a result, brain nerve cells are deprived of their oxygen supply and can begin to die within minutes. Lost brain cells do not regenerate and are replaced by fluid-filled cavities known as infarcts. In a stroke, some brain cells may be lost irreversibly and immediately. Other cells, for instance those around the ischemic focus, may suffer acute damage and remain in a compromised state for hours. It is also known in the art that brain damage may continue for days after the initial ischemic event.

Because patients do not often get to an emergency room immediately after the stroke event, new therapies are needed that are useful even when first administered at times following the event.

### SUMMARY

The present invention provides dosing regimens of EPO after an ischemic event as well as methods of treatment for a subject who has had such an event.

One embodiment of the present invention is a dosing regimen of erythropoietin for promoting recovery after an ischemic event comprising administering to a subject in need a therapeutically effective amount of EPO, wherein a first dose of EPO is delivered within about 8 to about 26 hours after the ischemic event followed by a second dose of EPO delivered within about 8 to about 26 hours after the first dose.

Another embodiment of the present invention is a method for treating a subject having an ischemic event comprising administering to said subject a therapeutically effective amount of EPO, wherein a first dose of EPO is delivered within about 8 to about 26 hours after the ischemic event followed by a second dose of EPO delivered within about 8 to about 26 hours after the first dose.

In a further embodiment, the present invention provides a method for promoting functional recovery in a subject after an ischemic event comprising administering to said subject a therapeutically effective amount of EPO, wherein a first dose of EPO is delivered within about 8 to about 26 hours after the ischemic event followed by a second dose of EPO delivered within about 8 to about 26 hours after the first dose.

In yet another aspect, the present invention also relates to a method for reducing infarct size in a subject having received an initial exposure to EPO within 6 hours of an ischemic event comprising administering to said subject an amount of EPO between about 1500 IU/kg to about 4500 IU/kg per dose, wherein a first dose of EPO is delivered within about 8 to about 26 hours after the initial exposure to EPO followed by a second dose of EPO delivered within about 8 to about 26 hours after the first dose.

In a further aspect, the present invention relates to a method for inhibiting apoptosis or inflammation in CNS in a subject after an ischemic event comprising administering to said subject a therapeutically effective amount of EPO, wherein a first dose of EPO is delivered within about 8 to about 26 hours after the ischemic event followed by a second dose of EPO delivered within about 8 to about 26 hours after the first dose.

In certain preferred embodiments of this invention, the first dose of EPO is delivered about 24 hours after the ischemic event. Also in certain preferred embodiments of this invention, the second dose is delivered at about 24 hours after the first dose. Preferably, the first dose of EPO is delivered about 24 hours after the ischemic event, and the second dose is delivered at about 48 hours after the ischemic event. Further, a third dose of EPO can be delivered within about 20 hours to about 60 hours after the ischemic event. Preferably, the third dose of EPO is delivered within about 8 to 24 hours after the second dose.

Preferred embodiments of this invention include dosing regimens and methods of treatment wherein each dose of EPO comprises a subcutaneous, intramuscular, intravenous, or intra-peritoneal injection of EPO.

Preferred embodiments of this invention also include dosing regimens and methods of treatment wherein each EPO dosage delivered is selected from about 500 IU/kg to about 10000 IU/kg. In one embodiment, particularly for reducing infarct size in a subject having received an initial exposure to EPO within 6 hours of an ischemic event, each EPO dosage delivered is selected from about 1500 IU/kg to about 4500 IU/kg. Preferably, each EPO dosage delivered is selected from about 1800 IU/kg to about 4000 IU/kg. More preferably, each EPO dosage delivered is selected from about 2000 IU/kg to about 3000 IU/kg. Most preferably, each EPO dosage delivered is about 2500 IU/kg. In another embodiment, each EPO dosage delivered is selected from about 2500 IU/kg to about 5000 IU/kg. Preferably, at least one EPO dosage delivered is about 2500 IU/kg. More preferably, each EPO dosage delivered is about 2500 IU/kg.

In more preferred embodiments of this invention, the ischemic event is a stroke. Particularly, the ischemic event is a CNS injury such as focal ischemic stroke or acute ischemic stroke.

Embodiments of this invention further include dosing regimens and methods of treatment wherein the erythropoietin is a long-acting EPO.

### BRIEF DESCRIPTION OF THE FIGURES

The accompanying figures illustrate several aspects of the invention. A brief description of the figures is as follows:

FIG. 1 shows that single day dosing has no effect on infarct size or functional outcome. Box-whisker graphs (A) and (B) demonstrate that Dextrorphan and EPO were ineffective at reducing the 7-day (A) infarct size or at improving (B) functional outcome, when given at the time of occlusion and again at 1 hour (hr) after occlusion compared to vehicle treated animals;

FIG. 2 shows that multiple-day dosing of erythropoietin decreases infarct size and improves functional outcome. Rats subjected to middle cerebral artery occlusion (MCAO) and treated with EPO at 0 hr, 24 hr and 48 hr after occlusion showed in graph (A) a statistically significant reduction in infarct volume at 2500 IU/kg, and in graph (B) a significant improvement in functional outcome at 2500 and 5000 IU/kg, compared to vehicle treated animals (* p<0.05; ** p<0.01; *** p<0.001);

FIG. 3 shows that delayed multiple-day administration of EPO improves functional outcome independent of decreasing infarct size. Administration of EPO at 6 hr, 24 hr, and 48 hr following MCAO had no effect on (A) infarct size, when given at either 2500 or 5000 IU/kg; however, (B) both dose levels significantly improved functional outcome (*p<0.05; **p<0.01);

FIG. 4 shows that EPO initiated as late as 24 hr following occlusion improves functional outcome. A dosing regimen that delayed the initial dose of EPO for a full 24 hr after occlusion followed by a second dose at 48 hr (A) was not effective at decreasing infarct size but (B) significantly improved functional outcome (*p<0.01).

### DETAILED DESCRIPTION

The embodiments of the present invention described below are not intended to be exhaustive or to limit the invention to the particular embodiments disclosed in the following detailed description. Rather, the embodiments are described so that others skilled in the art can understand the principles and practices of the present invention. If not specifically mentioned below, the disclosures of each patent, published patent application and publication referenced in the following description are hereby incorporated by reference in their entirety for any and all purposes.

The present invention provides a dosing regimen of erythropoietin for promoting recovery after an ischemic event comprising administering to a subject in need a therapeutically effective amount of EPO, wherein a first dose of EPO is delivered within about 8 to about 26 hours after the ischemic event followed by a second dose of EPO delivered within about 8 to about 26 hours after the first dose.

As used herein, an “ischemic event” occurs when a subject experiences a temporary or permanent reduction in blood flow and/or oxygen delivery in the central nervous system (CNS), potentially resulting in a damage such as necrosis or infarct of the non-perfused region. Ischemic events include, but are not limited to, acute CNS injury such as stroke, trauma such as a traumatic brain or spinal cord injury, transient ischemic attacks, infarct, ischemia-reperfusion injury, retinal damage, ischemia due to organ, tissue or cell transplantation or other surgical procedures. Particularly, for purposes of this invention, an ischemic event is a cerebral ischemic event, especially an interruption of cranial blood flow. More specifically, an ischemic event is a stroke, including but not limited to focal ischemic stroke or acute ischemic stroke.

As used herein, ischemia-reperfusion is a local interruption of blood flow to an organ, such as the brain, and subsequent restoration, usually abrupt, of blood flow.

The damage that results from acute ischemic stroke is dynamic. The injury evolves over several days following the initial insult. Multiple mechanisms contribute to an expanding area of neuronal cell and supportive cell death including the loss of ionic homeostasis, free radical damage, excitotoxicity, apoptosis and inflammation (1). Mechanisms of reconstruction and remodeling are active during the weeks to months following the initial injury in an attempt to compensate, to some degree, for the damage that has occurred (2). Both the events responsible for damage and those that contribute to recovery provide an opportunity for therapeutic intervention. A desirable treatment is one that can block the mechanisms that induce cell death or enhance the recovery processes or ideally, both. Potential treatment candidates are molecules with erythropoietic activity along the lines of the native hematopoietic cytokine Erythropoietin.

Recently, Erythropoietin has commanded considerable attention for its effects in non-hematopoietic systems including its function in the nervous system (4). In the central nervous system (CNS) Erythropoietin is produced and released locally by astrocytes in response to hypoxia (5, 6) while the Erythropoietin receptor has been localized to subtypes of neurons, as well as astrocytes and microglia (7, 8). The function of Erythropoietin in these cell types remains unclear but it has been shown to block programmed cell death in vitro, induced by a number of different stimuli including, glutamate, hypoxia (9), and serum withdrawal (10) suggesting that it may function to promote cell survival by blocking apoptosis (11). In addition to its neuroprotective effects, Erythropoietin has been reported to modulate inflammation (12), another potential target of stroke therapy (13). Furthermore, Erythropoietin has been shown to reduce the damage observed in animal models of CNS injury including models of, stroke (14), spinal cord injury (15), traumatic brain injury (14) and retinal damage (16) and has recently been implicated in the protective effects of ischemic preconditioning (6).

The function of Erythropoietin within the CNS has attracted considerable attention particularly due to its reported neuroprotective activity. The ability of Erythropoietin to limit damage in relevant models of CNS injury combined with data suggesting it has the potential to act on several mechanisms in the disease process makes Erythropoietin an attractive candidate to treat acute disorders of the nervous system including stroke. While the exact mechanism by which Erythropoietin elicits these protective effects is unclear, data that Erythropoietin decreases apoptosis in models of stroke (10), spinal cord injury (15), and retinal injury (16) suggests that its ability to block apoptosis is a critical function. Given that apoptosis can occur for days following an initial ischemic event, continued dosing with Erythropoietin in the days following the insult might be necessary to optimize the therapeutic effect. While to date, no pre-clinical studies have addressed this issue, continued administration of Erythropoietin over several days has been reported with positive results in a pilot clinical study to assess Erythropoietin in treating ischemic stroke (19).

Natural or native Erythropoietin is a 30-kDa glycoprotein that controls erythropoiesis by regulating the differentiation, proliferation and survival of erythroid precursor cells (3). As used herein and as defined within the claims, the term “EPO” shall include those polypeptides and proteins that have the capacity to stimulate erythropoiesis as mediated through the native Erythropoietin receptor. The term “EPO” includes natural or native erythropoietin as well as recombinant human erythropoietin (r-HuEPO). Also included within the scope of the term EPO are erythropoietin analogs, erythropoietin isoforms, erythropoietin mimetics, erythropoietin fragments, hybrid erythropoietin proteins, fusion protein oligomers and multimers of the above, homologues of the above, glycosylation pattern variants of the above, peptide mimetics and muteins of the above, and further regardless of the method of synthesis or manufacture thereof including, but not limited to, recombinant (whether produced from cDNA or genomic DNA), synthetic, transgenic, and gene activated methods, and further those Erythropoietin molecules containing the minor modifications enumerated above. Methods of designing and synthesizing, e.g., peptide mimetics are well known to those of ordinary skill in the art and are described, e.g., in U.S. Pat. Nos. 4,833,092, 4,859,765; 4,853,871 and 4,863,857 the disclosures of each of which are hereby incorporated by reference herein in their entirety and for all purposes. In addition to polypeptides and proteins having erythropoietic activity, small molecules capable of promoting erythropoiesis are also within the scope of the term EPO and include, for example, compounds with erythropoietin activity, such as molecules that stimulate erythropoietin production through upstream activation events.

Particularly preferred EPO molecules are those that are capable of stimulating erythropoiesis in a mammal. Specific examples of erythropoietin include, Epoetin alfa (EPREX®, ERYPO®, PROCRIT®), novel erythropoiesis stimulating protein (NESP™, ARANESP™ and darbepoetin alfa) such as the hyperglycosylated analog of recombinant human erythropoietin (Epoetin) described in European patent application EP640619. Other EPO molecules contemplated within the scope of the invention include human erythropoietin analogs (such as the human serum albumin fusion proteins described in the international patent application WO 99/66054), erythropoietin mutants described in the international patent application WO 99/38890, erythropoietin omega, which may be produced from an Apa I restriction fragment of the human erythropoietin gene described in U.S. Pat. No. 5,688,679, altered glycosylated human erythropoietin described in the international patent application WO 99/11781 and EP1064951, PEG conjugated erythropoietin analogs described in WO 98/05363, WO 01/76640, or U.S. Pat. No. 5,643,575. Specific examples of cell lines modified for expression of endogenous human erythropoietin are described in international patent applications WO 99/05268 and WO 94/12650. The generally preferred form of EPO is purified recombinant human EPO (r-HuEPO), currently formulated and distributed under the trademarks of EPREX®, ERYPO®, PROCRIT® or ARANESP™. The disclosures of each of the patents and published patent applications mentioned in this paragraph are hereby incorporated by reference herein for any and all purposes.

Long-acting forms of EPO are also contemplated and may be preferred in some embodiments of the present invention for administration as the second or third exposure in a dosing segment. As used herein, a “long-acting EPO” includes sustained-release compositions and formulations of EPO with increased circulating half-life, typically achieved through modification such as reducing immunogenicity and clearance rate, and EPO encapsulated in polymer microspheres. Examples of “long-acting EPO” include, but are not limited to, conjugates of erythropoietin with polyethylene glycol (PEG) disclosed in PCT publication WO 2002049673 (Burg et al.), PEG-modified EPO disclosed in PCT publication WO 02/32957 (Nakamura et al.), conjugates of glycoproteins having erythropoietic activity and having at least one oxidized carbohydrate moiety covalently linked to a non-antigenic polymer disclosed in PCT publication WO 94/28024 (Chyi et al.), and other PEG-EPO prepared using SCM-PEG, SPA-PEG AND SBA-PEG. The disclosures of each of these published patent applications are hereby incorporated by reference herein in their entirety and for all purposes.

The preferred polyethylene glycol moieties are methoxy polyethylene glycol (mPEG) moieties. The moieties are preferably added using succinimidyl ester derivatives of the methoxy polyethylene glycol species. In one example a preferred succinimidyl ester derivative of a methoxy polyethylene glycol species includes: succinimidyl esters of carboxymethylated polyethylene glycol (SCM-PEG) of the following formula,

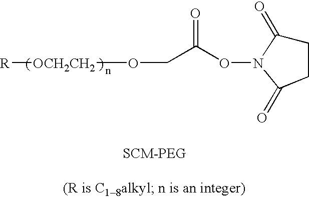

succinimidyl derivatives of poly(ethylene glycol) propionic acid (SPA-PEG) of the following formula, wherein R is C₁₋₈alkyl and n is an integer, (R—(OCH₂CH₂)ₙ—O—CH₂CH₂—CO—OSu); and succinimidyl derivatives of poly(ethylene glycol) butanoic acid (SBA-PEG) of the following formula, wherein R is C₁₋₈alkyl and n is an integer, (R—(OCH₂CH₂)ₙ—O—CH₂CH₂CH₂—CO—OSu).

Methods to prepare SCM-PEG, SPA-PEG, and SBA-PEG are well known in the art. For example, U.S. Pat. No. 5,672,662 to Harris et al. describes active esters of PEG acids and related polymers that have a single propionic or butanoic acid moiety and no other ester linkages. Preparation of SCM-PEG has been described in, for example, Veronese et al. (1989), Journal of Controlled Release, 110: 145-54.

SPA-PEG includes mPEG-SPA (methoxy-PEG-Succinimidyl Propionate). SBA-PEG includes mPEG-SBA (methoxy-PEG-Succinimidyl Butanoate). Activated polymers such as SBA-PEG and SPA-PEG, are both commercially available and may be obtained from, e.g., Shearwater Polymers, Inc., Huntsville, Ala., U.S.A.

SCM-PEG (R—(OCH₂CH₂)ₙ—O—CH₂—CO—OSu; R is C₁₋₈alkyl and n is an integer) includes methoxy-PEG-succinimidyl ester of carboxymethylated PEG (mPEG-SCM). According to Greenwald et al., SCM-PEG “reaction with protein would form a stable amide, but t1/2 hydrolysis has been reported [Shearwater Polymers, Huntsville, Ala., January 1996 catalog, p 46] as <1 min at pH 8, thus minimizing its usefulness for protein modification in aqueous solution . . . . ” (Bioconjugate Chem., 7 (6), 638 -641, 1996).

At present, SCM-PEG may be custom synthesized by, e.g., Delmar Chemicals, Inc, Quebec, Canada.

SCM-PEG, SPA-PEG and SBA-PEG react primarily with the primary amino groups of lysine and the N-terminal amino group. Reactions with EPO are shown below for SCM-PEG5K, SPA-PEG5K and SBA-PEG5K, respectively, wherein OSu represents n-hydroxysuccinimide, and m is 1-4, n is an integer:

(SCM-PEG) CH₃O—(OCH₂CH₂)N—O—CH₂—CO-OSU+EPO (NH₂)_{M}→CH₃O—(OCH₂CH₂)_{N}—O—CH₂—CO—NH-EPO

(SPA-PEG) CH₃O—(OCH₂CH₂)_{N}—O—CH₂CH₂—CO-OSU+EPO (NH₂)_{M}→CH₃O—(OCH₂CH₂)_{N}—O—CH₂CH₂—CO—NH-EPO

(SBA-PEG)CH₃O—(OCH₂CH₂)_{N}—O—CH₂CH₂CH₂—CO—OSU+EPO (NH₂)_{M}→CH₃O—(OCH₂CH₂)_{N}—O—CH₂CH₂CH₂—CO—NH-EPO

To explore the hypothesis that Erythropoietin may be a useful therapeutic for acute injuries to the CNS including stroke and may potentially inhibit processes responsible for delayed neurotoxicity such as apoptosis and inflammation, Applicants tested several dosing regimens in an attempt to determine the optimum activity of Erythropoietin in a model of focal ischemic stroke. Applicants show herein that multiple doses administered over several days can reduce the infarct size and improve the functional outcome of rats subject to permanent middle cerebral artery occlusion (MCAO). In addition, delayed administration of Erythropoietin, even up to about 24 hr after occlusion, can improve functional outcome independent of a reduction in infarct size.

Applicants have very discovered that a multiple day dosing regimen with 2500 IU/kg Erythropoietin was the most effective at improving the outcome of rats subjected to MCAO. In these studies the observed decrease in infarct size was relatively modest (30%), this would not be surprising for a molecule that has an anti-apoptotic mechanism of action since necrosis is thought to be the primary method of cell death contributing to the infarct volume in a permanent model of focal ischemic stroke (20).

The present invention thus provides a method for treating a subject having an ischemic event comprising administering to said subject a therapeutically effective amount of Erythropoietin, wherein a first dose of Erythropoietin is delivered within about 8 to about 26 hours after the ischemic event followed by a second dose of Erythropoietin delivered within about 8 to about 26 hours after the first dose. Particularly, treatment of a subject having an ischemic event includes promoting recovery from any consequence of an ischemic event, such as neurological lesions or infarcts. In one aspect, a recovery from an ischemic event is indicated by a decrease in the infarct size. In another aspect, a recovery from an ischemic event is indicated by an improvement in the functional outcome of the patient, such as an improvement in one or more scores obtained from determined by a behavioral scoring system. The result of the treatment provided by the present invention can be evaluated using methods of assessment well known in the art, including, but not limited to, a medical imaging system such as Magnetic Resonance Imaging (MRI), CTA (Computed Tomography Angiography) and CT (Computed Tomography) scan, a neurological test, a whisker touch test, or a foot fault test (19).

The present invention also provides a method for inhibiting apoptosis or inflammation in CNS in a subject after an ischemic event comprising administering to the subject a therapeutically effective amount of Erythropoietin, wherein a first dose of Erythropoietin is delivered within about 8 to about 26 hours after the ischemic event followed by a second dose of Erythropoietin delivered within about 8 to about 26 hours after the first dose.

The results demonstrated that early administration of Erythropoietin is necessary to achieve a reduction in infarct size. The effect was lost when treatment was initiated 6 hr after occlusion. Particularly, multiple daily doses of Erythropoietin given at the time of occlusion and at 24 hr and 48 hr post occlusion at a dose of 2500 IU/kg resulted in a 30% decrease in infarct size, which was not observed at either the 5000 IU/kg or 1250 IU/kg dose levels. Therefore, the present invention provides a method for reducing infarct size in a subject having received an initial exposure to Erythropoietin within 6 hours of an ischemic event comprising administering to said subject an amount of Erythropoietin between about 1500 IU/kg to about 4500 IU/kg per dose, wherein a first dose of Erythropoietin is delivered within about 8 to about 26 hours after the initial exposure to Erythropoietin followed by a second dose of Erythropoietin delivered within about 8 to about 26 hours after the first dose.

As used herein, the term “exposure” refers to a single dose, repeated individual doses, or dosing as may be provided relatively continuously after a single administration, e.g., of a long-acting EPO, application, e.g., of a transdermal patch comprising EPO, or implantation of an EPO implant.

In addition, Applicants have now surprisingly discovered significant functional improvements in rats subjected to permanent MCAO when EPO was given about 24 hr following the insult. Thus, the present invention further provides a method for promoting functional recovery in a subject after an ischemic event comprising administering to said subject a therapeutically effective amount of EPO, wherein a first dose of EPO is delivered within about 8 to about 26 hours after the ischemic event followed by a second dose of EPO delivered within about 8 to about 26 hours after the first dose.

In a related embodiment of this invention, EPO can be administered to the patient as a third dose. The third dose can be administered preferably within about 20 hours to about 60 hours after the ischemic event. Where a third dose is given it can be delivered within about 8 to about 24 hours after the second dose.

As used herein, the term “functional recovery” refers to a behavioral improvement in a subject after an ischemic event. Functional recovery in an animal can be evaluated, for example, using a modified Hernandez-Schallert foot-fault test (18), wherein the animal is placed on a grid work with 2 cm spaces between 0.5 cm diameter metal rods and observed for two minutes, during which the numbers of times their front and hind limbs fall through the spaces are counted. Functional recovery in humans may be evaluated by instruments designed to measure elemental neurological functions such as motor strength, sensation and coordination, cognitive functions such as memory, language and the ability to follow directions, and functional capacities such as basic activities of daily living or instrumental activities. Recovery of elemental neurological function can be measured with instruments such as the NIH Stroke Scale (NIHSS) (31), recovery of cognitive function can be measured with neuropsychological tests such as Boston Naming Test, Trail-making Tests, and California Verbal Learning Test, and activities of daily living may be measured with instruments such as the ADCS/ADL (Alzheimer's Disease Clinical Studies/Activities of Daily Living) scale or the Bristol Activities of Daily Living Scale, all tests and scales known in the art.

The delayed dosing paradigm, as described herein, did not result in a decrease in infarct size. One possible explanation for this observation is that TTC (2,3,5-triphenyltetrazolium chloride) staining may not be accurately representing the infarct area. To address this possibility a more comprehensive morphological analysis was performed on sections from representative animals and compared to TTC analysis. No intact neurons were detected within the border of the TTC staining (data not shown) suggesting that this phenomenon was not responsible for our observation. Delayed administration of EPO, therefore, improves function independent of decreasing the size of the infarcted area perhaps by inhibiting delayed apoptosis in areas distant from the infarct area or possibly by enhancing endogenous reconstruction and remodeling events.

As mentioned previously, apoptosis can occur for several days following an ischemic insult. Delayed apoptosis occurs in the ischemic penumbra and contributes to the final infarct volume (21, 22). It also occurs in areas distant from the infarct area most likely due to the loss of trophic support for neurons that have lost key projections into the infarcted area. Since EPO has been shown to be trophic for neuronal populations (10, 23), a possible explanation for the effects of delayed EPO administration is the prevention of delayed apoptosis of neurons located anatomically some distance from the infarct area. The sparing of these populations of neurons may explain the preservation of functional performance observed in our behavioral evaluation.

Another explanation that cannot be excluded is that EPO enhances the functional recovery process by supporting such reconstruction and remodeling mechanisms as neurite outgrowth, synapse formation, synapse strengthening or unmasking. The improvement in function observed as early as 7 days argues against the establishment of new long-distance functional connections, however, two reports suggest that at least the beginnings of plasticity are evident by 7 days. Kawamate et al. (24) examining the effect of basic fibroblast growth factor (bFGF) in a rat model of focal ischemic stroke and Stroemer et al., (25) looking at d-amphetamine treatment in a similar model, observed an improvement in functional performance with drug treatment without a concomitant decrease in infarct size. Notably, while the animals were observed for up to two months in these studies, the behavioral improvement was significant by 7 days.

Immunohistochemical analysis revealed evidence of increased neurite sprouting in both studies, determined by an increase in growth associated protein 43 (GAP43) expression, which peaked at 3 days following occlusion. EPO has been shown previously to promote neurite sprouting (26). Taken together these data suggest that some repair processes may be initiated within the first few days following injury and that EPO could be acting on these early processes to enhance functional recovery. Other reorganization processes such as unmasking or synaptic strengthening could occur over much shorter time periods but EPO's effects on these events has not been shown.

Those of ordinary skill in the art are readily capable of determining appropriate amounts and manners of dosing in various circumstances. The amount of EPO to be administered in any particular exposure of any given dosing segment is not particularly limited, and any amount of EPO may be administered per exposure, dosing segment, or multiple day dosing regimen so long as substantially no toxic effects due to administration of EPO are manifested. That being said, and only for the purpose of providing additional guidance and not being unnecessarily bound thereto, general therapeutic guidelines suggest that subjects would desirably receive in each dose of EPO a therapeutically effective amount from about 500 IU/kg to about 10000 IU/kg. Preferably, a subject would receive in each dose of EPO an amount from about 1250 IU/kg to about 5000 IU/kg. More preferably, a subject would receive in each dose of EPO an amount from about 2500 IU/kg. As mentioned above, the dosing of EPO can be provided in any known, or newly developed, dosing format.

The invention contemplates that a variety of routes of administration of EPO could be used in the practice of this invention. Preferably, each dose of EPO may desirably be provided in a format that can provide patient exposure to EPO as quickly as possible. That said, a variety of administration routes are contemplated, including, but not limited to intravenous dosing, subcutaneous dosing, intramuscularly, or intraperitoneal. To the extent that oral formulations are or would become available, such avenues of administration are also considered and my be particularly suited for the second or subsequent EPO dosings as described herein.

Subjects that may benefit from the dosing regimen are not particularly limited and may include both human and animal subjects, preferably mammalian subjects.

The following example is provided to illustrate the present invention, and should not be construed as limiting thereof. This invention will be better understood by reference to the figures and examples that follow, but those skilled in the art will readily appreciate that these are only illustrative of the invention as described more fully in the claims which follow thereafter.

### EXAMPLE 1

### Materials and Methods

Animals and MCAO Surgery

Procedures involving animals were approved by the Johnson & Johnson Institutional Animal Care and Use Committee. Male Sprague-Dawley rats (Charles River Laboratories, Raleigh, N.C.) weighing between 290 g and 320 g were housed individually in a temperature-controlled environment on a 12 hr (6:00-18:00) light-dark cycle and given food and water ad libidum. Animals were anesthetized with 2.5% isoflurane and the body temperature maintained at 37° C. Permanent MCAO was performed essentially as described by Zimmerman et al. (17). Briefly, an incision was made ventrally and both the left and right common carotid arteries (CCA) were visualized. The right CCA was permanently occluded using a 4-0 silk ligature. A craniotomy was performed 1 mm anterior and 3-4 mm lateral to the foramen ovale to visualize the right middle cerebral artery (MCA). The MCA was then occluded permanently by cauterization distal to the lenticulostriate arteries. The left CCA was then occluded transiently using an aneurysm clip for a period of 1 hour. Following occlusion, incisions were closed with surgical staples and Bupivicane (0.25%) was applied to the incision site. Animals were recovered from anesthesia under a warming lamp.

Twenty-four hours later rats were evaluated using a neurological test, a whisker touch test and a foot fault test. As shown in Table 1, rats subjected to MCAO were assessed 24 hr after occlusion for injury severity. Three tests were performed: a neurological assessment, a whisker touch test and a foot-fault test (19). Animals that scored outside pre-determined parameters were judged to have a deficit that was either too mild or too severe and were excluded from further analysis. Animals that scored a 0 on the neurological score or had a whisker touch score of ≧1 AND a foot fault score of ≦2 (no injury) or animals that had a neurological score of ≧2 (severe injury) were excluded from further analysis.

Animals were analyzed at 7 days post occlusion for infarct size and functional outcome.

TABLE 1
Behavioral scoring system for injury assessment.

| Test | Criteria | Score |
|---|---|---|
| Neurological | No deficit | 0 |
| Score | Failure to extend effected forepaw | 1 |
| | Circling to effected side | 2 |
| | Falling toward effected side | 3 |
| | No spontaneous walking | 4 |
| Whisker Touch | No response | 0 |
| | Response >2 sec | 1 |
| | Normal response (no delay) | 2 |
| Foot Fault | Effected side limb falls through grid | # of occurrences |

Drugs and Administration

Dextrorphan tartarate (Sigma Chemicals) was dissolved in EPO's vehicle (4.38 mg/ml NaCl, 1.1 mg/ml NaH₂PO₄, 1.6 mg/ml Na₂HPO₄, 5.0 mg/ml Glycine, 0.3 mg/ml, Tween 80 in Distilled Water, Ph 6.9) at a concentration of 25 mg/ml. Recombinant Human Erythropoietin (Epoetin alfa) was diluted to its final concentration in vehicle solution. All drugs were administered intravenously (i.v.) through the tail vein at a volume of 1 ml/kg by one of three dosing regimens:

1) One-Day Dosing Regimen—Vehicle, Dextrorphan (25 mg/kg) or EPO (5000 IU/kg) was administered immediately prior to the occlusion of the left CCA (0 hr), followed by a second dose given 1 hr later, immediately after the restoration of blood flow.

2) Multiple-Day Dosing Regimen—Vehicle, or EPO (5000 IU/kg, 2500 IU/kg or 1250 IU/kg) was administered at 0 hr with subsequent doses given 24 hr and 48 hr later.

3) Delayed Multiple-Day Dosing Regimen—Vehicle or EPO (5000 IU/kg or 2500 IU/kg) was given at 6 hr, 24 hr, and 48 hr after occlusion or at 24hr and 48hr post occlusion.

Determination of Infarct Volume

Animals were sacrificed after 7 days and their brains removed and placed in ice cold PBS. Brains were cut into 2 mm thick sections using a brain matrix (Kent Scientific), then stained with 2% 2,3,5-triphenyltetrazolium chloride (TTC). Brain sections were imaged using a CCD (Charge-Coupled Device) camera connected to a stereomicroscope and infarct size determined with computer assistance. The infarct volume of all sections was combined to provide the absolute infarct volume. The relative infarct volume (absolute infarct volume/total volume of the cerebral hemisphere) was also determined.

Behavioral Analysis

Animals were evaluated for functional recovery using a modified Hernandez-Schallert foot-fault test (18). In this test, an animal is placed on a grid work with 2 cm spaces between 0.5 cm diameter metal rods. The animal is observed for two minutes and the numbers of times their front and hind limbs fall through the spaces are counted.

Statistical Analysis

A non-parametric analysis of the treatment groups was performed using the Kruskal-Walis test, followed by a post-hoc analysis to compare treatment groups to the vehicle control group.

### Results

Surgical Outcome

A total of 229 animals underwent MCAO for this study. Of these 28 died prior to analysis at 7 days and 4 were excluded upon 24 hr evaluation (see Materials and Methods and Table 1). In an attempt to control for infarct size and infarct location in this study, animals that had an infarct with sub-cortical involvement (n-21) were excluded from analysis. There was no statistical difference between treatment groups regarding body weight or body temperature.

Single Day Dosing Regimen

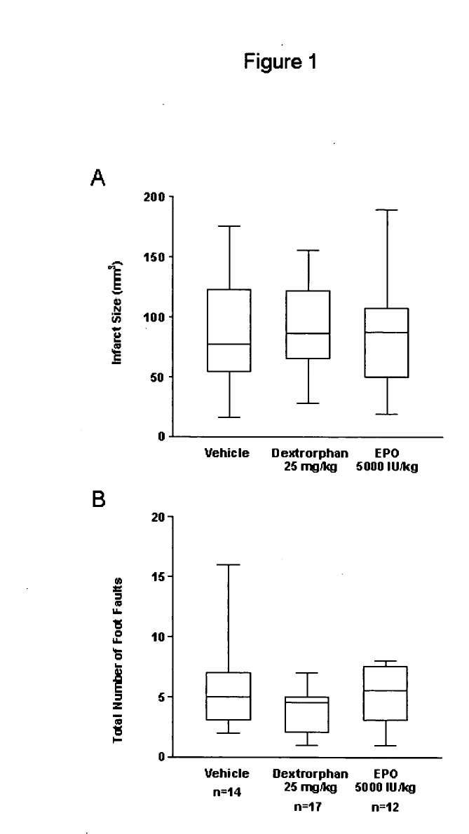

The ability of a single day dosing regimen of EPO to decrease infarct volume and improve functional outcome was tested. Animals treated with Dextrorphan (25 mg/kg) or EPO (5006 IU/kg) showed no difference in infarct volume or functional outcome when analyzed 7 days post occlusion compared to vehicle treated animals (FIG. 1). Dextrorphan decreased the infarct volume when analyzed at 24 hr post occlusion (data not shown).

Multiple Day Dosing Regimen

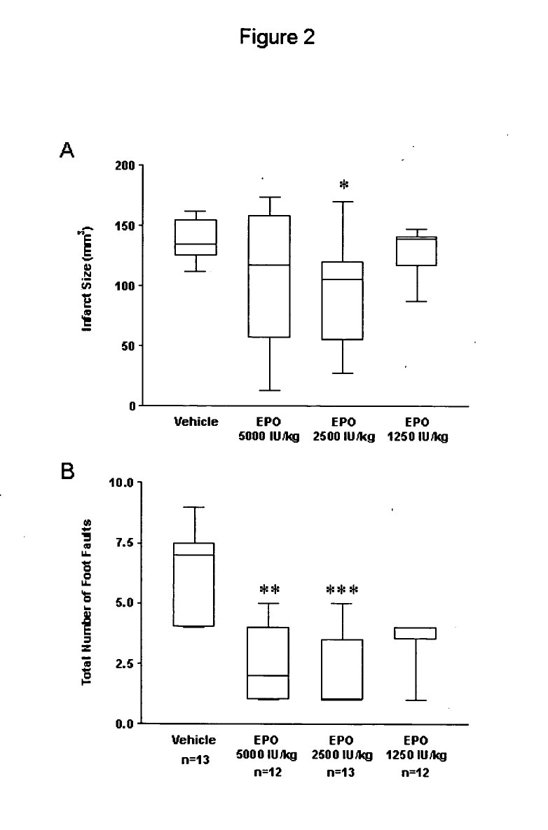

The effect of multiple daily doses of EPO given at the time of occlusion and at 24 hr and 48 hr post occlusion was also tested. Multiple day treatment with EPO at a dose of 2500 IU/kg resulted in a 30% decrease in both the absolute infarct size (FIG. 2A) and the relative infarct size (data not shown) that was statistically significant (p<0.05). This decrease in infarct size was not observed at either the 5000 IU/kg or 1250 IU/kg dose levels. Behavioral analysis showed a significant improvement in functional recovery of 61% at 5000 IU/kg and 65% at 2500 IU/kg (p<0.01 and p<0.001 respectively, FIG. 2B) compared to vehicle treated animals. EPO at 1250 IU/kg had no effect on functional outcome.

Delayed-Multiple-Day Dosing Regimen

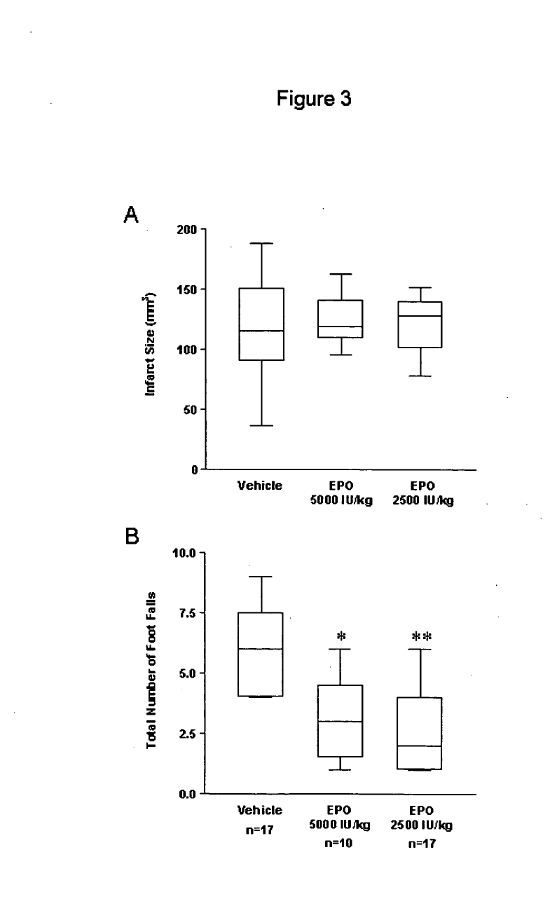

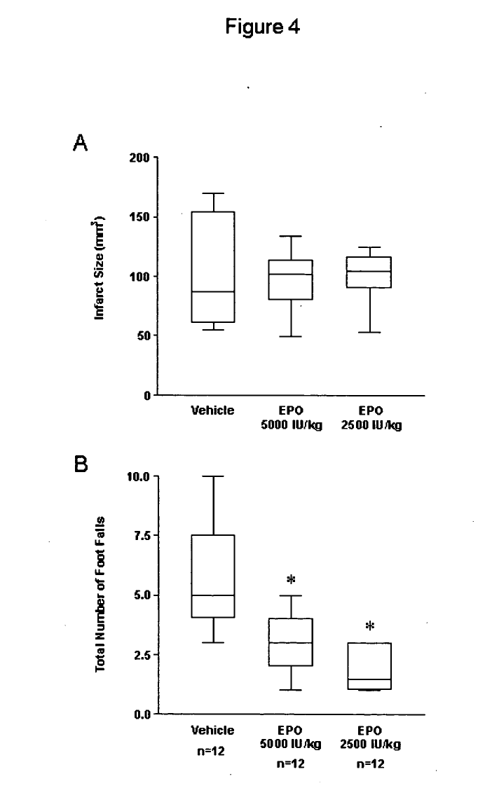

Due to the well-known problems in getting stroke patients to emergency rooms in a timely fashion, it is important to determine the time window required before initiating therapy. To identify the time window for the effects of EPO in our MCAO model, the initial dose of EPO was delayed 6 hr after occlusion and then followed with doses at 24 hr and 48 hr. With a 6 hr delay, neither 5000 IU/kg nor 2500 IU/kg doses of EPO resulted in a significant decrease in infarct size (FIG. 3). Unexpectedly, both the 5000 IU/kg and the 2500 IU/kg dose levels increased the functional score by 46% (p<0.05) and 56% (p<0.01) over vehicle, respectively. When the first day dose was eliminated and EPO was given at 24 hr and 48 hr following injury similar results were observed (FIG. 4) i.e. no decrease in infarct size and a significant improvement in functional outcome at 5000 IU/kg (49%) and 2500 IU/kg (68%) over vehicle (p<0.01).

A multiple day dosing regimen of EPO reduced the infarct size and improved functional outcome compared to vehicle treated animals. Delaying the initial dose of EPO up to 24 hr post-occlusion was effective at improving functional outcome but not at decreasing infarct size. The single day dosing regimen had no effect on infarct size or functional outcome.

This work supports the previously reported efficacy of EPO in animal models of acute ischemic stroke. Furthermore, the data provides evidence for extending the therapeutic time window available for EPO to improve functional outcome, and demonstrates that dosing regimen is important when considering EPO as a therapy for disorders of the nervous system.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

### References

1. Dimagl U, Iadecola C, Moskowitz M A. Pathobiology of ischaemic stroke: An integrated view. Trends Neurosci. 1999;22:391-397

2. Chen R, Cohen L G, Hallett M. Nervous system reorganization following injury. Neuroscience. 2002;111:761-773

3. Silva M, Grillot D, Benito A, Richard C, Nunez G, Fernandez-Luna J L. Erythropoietin can promote erythroid progenitor survival by repressing apoptosis through bcl-xl and bcl-2. Blood. 1996;88:1576-1582

4. Cerami A, Brines M L, Ghezzi P, Cerami C J. Effects of epoetin alfa on the central nervous system. Semin Oncol. 2001;28:66-70.

5. Masuda S, Okano M, Yamagishi K, Nagao M, Ueda M, Sasaki R. A novel site of erythropoietin production. Oxygen-dependent production in cultured rat astrocytes. J Biol Chem. 1994;269:19488-19493

6. Ruscher K, Freyer D, Karsch M, Isaev N, Megow D, Sawitzki B, Priller J, Dimagl U, Meisel A. Erythropoietin is a paracrine mediator of ischemic tolerance in the brain: Evidence from an in vitro model. J Neurosci. 2002;22:10291-10301.

7. Siren A L, Knerlich F, Poser W, Gleiter C H, Bruck W, Ehrenreich H. Erythropoietin and erythropoietin receptor in human ischemic/hypoxic brain. Acta Neuropathol (Berl). 2001;101:271-276

8. Nagai A, Nakagawa E, Choi H B, Hatori K, Kobayashi S, Kim S U. Erythropoietin and erythropoietin receptors in human cns neurons, astrocytes, microglia, and oligodendrocytes grown in culture. J Neuropathol Exp Neurol. 2001;60:386-392

9. Morishita E, Masuda S, Nagao M, Yasuda Y, Sasaki R. Erythropoietin receptor is expressed in rat hippocampal and cerebral cortical neurons, and erythropoietin prevents in vitro glutamate-induced neuronal death. Blood. 1997;89:55-64

10. Siren A L, Fratelli M, Brines M, Goemans C, Casagrande S, Lewczuk P, Keenan S, Gleiter C, Pasquali C, Capobianco A, Mennini T, Heumann R, Cerami A, Ehrenreich H, Ghezzi P. Erythropoietin prevents neuronal apoptosis after cerebral ischemia and metabolic stress. Proc Natl Acad Sci USA. 2001;98:4044-4049.

11. Wen T C, Sadamoto Y, Tanaka J, Zhu P X, Nakata K, Ma Y J, Hata R, Sakanaka M. Erythropoietin protects neurons against chemical hypoxia and cerebral ischemic injury by up-regulating bcl-xl expression. J Neurosci Res. 2002;67:795-803

12. Gorio A, Gokmen N, Erbayraktar S, Yilmaz O, Madaschi L, Cichetti C, Di Giulio A M, Vardar E, Cerami A, Brines M. Recombinant human erythropoietin counteracts secondary injury and markedly enhances neurological recovery from experimental spinal cord trauma. Proc Natl Acad Sci USA. 2002;99:9450-9455.

13. Priller J, Dirnagl U. Inflammation in stroke—a potential target for neuroprotection? Ernst Schering Res Found Workshop. 2002:133-157

14. Brines M L, Ghezzi P, Keenan S, Agnello D, de Lanerolle N C, Cerami C, Itri L M, Cerami A. Erythropoietin crosses the blood-brain barrier to protect against experimental brain injury. Proc Natl Acad Sci USA. 2000;97:10526-10531.

15. Celik M, Gokmen N, Erbayraktar S, Akhisaroglu M, Konakc S, Ulukus C, Genc S, Genc K, Sagiroglu E, Cerami A, Brines M. Erythropoietin prevents motor neuron apoptosis and neurologic disability in experimental spinal cord ischemic injury. Proc Natl Acad Sci USA. 2002;99:2258-2263.

16. Junk A K, Mammis A, Savitz S I, Singh M, Roth S, Malhotra S, Rosenbaum P S, Cerami A, Brines M, Rosenbaum D M. Erythropoietin administration protects retinal neurons from acute ischemia-reperfusion injury. Proc Natl Acad Sci USA. 2002;99:10659-10664.

17. Zimmerman G A, Meistrell M, 3rd, Bloom O, Cockroft K M, Bianchi M, Risucci D, Broome J, Farmer P, Cerami A, Vlassara H, et al. Neurotoxicity of advanced glycation endproducts during focal stroke and neuroprotective effects of aminoguanidine. Proc Natl Acad Sci USA. 1995;92:3744-3748

18. Hernandez T D, Schallert T. Seizures and recovery from experimental brain damage. Exp Neurol. 1988;102:318-324

19. Ehrenreich H, Hasselblatt M, Dembowski C, Cepek L, Lewczuk P, Stiefel M, Rustenbeck H H, Breiter N, Jacob S, Knerlich F, Bohn M, Poser W, Ruther E, Kochen M, Gefeller O, Gleiter C, Wessel T C, De Ryck M, Itri L, Prange H, Cerami A, Brines M, Siren A L. Erythropoietin therapy for acute stroke is both safe and beneficial. Mol Med. 2002;8:495-505.

20. Leist M, Nicotera P. Apoptosis, excitotoxicity, and neuropathology. Exp Cell Res. 1998;239:183-201

21. Du C, Hu R, Csernansky C A, Hsu C Y, Choi D W. Very delayed infarction after mild focal cerebral ischemia: A role for apoptosis? J Cereb Blood Flow Metab. 1996;16:195-201

22. Ferrer I, Planas A M. Signaling of cell death and cell survival following focal cerebral ischemia: Life and death struggle in the penumbra. J Neuropathol Exp Neurol. 2003;62:329-339

23. Studer L, Csete M, Lee S H, Kabbani N, Walikonis J, Wold B, McKay R. Enhanced proliferation, survival, and dopaminergic differentiation of cns precursors in lowered oxygen. J Neurosci. 2000;20:7377-7383.

24. Kawamata T, Dietrich W D, Schallert T, Gotts J E, Cocke R R, Benowitz L I, Finklestein S P. Intracistemal basic fibroblast growth factor enhances functional recovery and up-regulates the expression of a molecular marker of neuronal sprouting following focal cerebral infarction. Proc Natl Acad Sci USA. 1997;94:8179-8184

25. Stroemer R P, Kent T A, Hulsebosch C E. Enhanced neocortical neural sprouting, synaptogenesis, and behavioral recovery with d-amphetamine therapy after neocortical infarction in rats. Stroke. 1998;29:2381-2393; discussion 2393-2385

26. Campana W M, Misasi R, O'Brien J S. Identification of a neurotrophic sequence in erythropoietin. Int J Mol Med. 1998;1:235-241.

27. Brewitt B A, U.S. Pat. No. 6,485,480, Treatment methods using homeopathic preparations of growth factors.

28. Isner et al., U.S. Pat. No. 5,980,887, Methods for enhancing angiogenesis with endothelial progenitor cells.

29. Ehrenreich et al, WO00/35475, Method for the treatment of cerebral ischaemia and use of erythropoietin or erythropoietin derivatives for the treatment of cerebral ischaemia.

30 Isner J M, U.S. Pat. No. 6,121,246, Method for treating ischemic tissue.

31. NIH Stroke Scale (NIHSS), Mar. 16, 2004, available on the web site of Internet Stroke Center at http://www.strokecenter.org/trials/scales/nihss.html.

What is claimed is:

1. A dosing regimen of erythropoietin for promoting recovery after an ischemic event comprising administering to a subject in need a therapeutically effective amount of EPO, wherein a first dose of EPO is delivered within about 8 to about 26 hours after the ischemic event followed by a second dose of EPO delivered within about 8 to about 26 hours after the first dose.

2. The dosing regimen of claim 1, wherein the first dose of EPO is delivered about 24 hours after the ischemic event.

3. The dosing regimen of claim 2, wherein the second dose is delivered at about 24 hours after the first dose.

4. The dosing regimen of claim 1, further comprising administering to the subject a third dose of EPO delivered within about 20 hours to about 60 hours after the ischemic event.

5. The dosing regimen of claim 4, wherein the third dose of EPO is delivered within about 8 to 24 hours after the second dose.

6. The dosing regimen of claim 1 wherein at least one dose of EPO is delivered by a subcutaneous, intramuscular, intravenous, or intra-peritoneal route of administration.

7. The dosing regimen of claim 1 wherein each EPO dosage delivered is selected from about 500 IU/kg to about 10000 IU/kg.

8. The dosing regimen of claim 7, wherein each EPO dosage delivered is selected from about 2500 IU/kg to about 5000 IU/kg.

9. The dosing regimen of claim 7, wherein each EPO dosage delivered is about 2500 IU/kg.

10. The dosing regimen of claim 1 wherein the ischemic event is a stroke.

11. The dosing regimen of claim 10, wherein at least one EPO dosage delivered is about 2500 IU/kg.

12. The dosing regimen of claim 11, wherein each EPO dosage delivered is about 2500 IU/kg.

13. The dosing regimen of claim 1 wherein the erythropoietin is a long-acting EPO.

14. A method for treating a subject having an ischemic event comprising
administering to said subject a therapeutically effective amount of EPO, wherein a first dose of EPO is delivered within about 8 to about 26 hours after the ischemic event followed by a second dose of EPO delivered within about 8 to about 26 hours after the first dose.

15. The method of claim 14, wherein the first dose of EPO is delivered about 24 hours after the ischemic event.

16. The method of claim 15, wherein the second dose is delivered at about 24 hours after the first dose.

17. The method of claim 14, further comprising administering to the subject a third dose of EPO delivered within about 20 hours to about 60 hours after the ischemic event.

18. The method of claim 16, wherein the third dose of EPO is delivered within about 8 to 24 hours after the second dose.

19. The method of claim 14, wherein each dose of EPO comprises a subcutaneous, intramuscular, intravenous, or intra-peritoneal injection of EPO.

20. The method of claim 14, wherein each EPO dosage delivered is selected from about 500 IU/kg to about 10000 IU/kg.

21. The method of claim 20, wherein each EPO dosage delivered is selected from about 2500 IU/kg to about 5000 IU/kg.

22. The method of claim 21, wherein each EPO dosage delivered is about 2500 IU/kg.

23. The method of claim 14, wherein the ischemic event is a stroke.

24. The method of claim 23, wherein at least one EPO dosage delivered is about 2500 IU/kg.

25. A method for promoting functional recovery in a subject after an ischemic event comprising administering to said subject a therapeutically effective amount of EPO, wherein a first dose of EPO is delivered within about 8 to about 26 hours after the ischemic event followed by a second dose of EPO delivered within about 8 to about 26 hours after the first dose.

26. The method of claim 25, wherein the first dose of EPO is delivered about 24 hours after the ischemic event.

27. The method of claim 26, wherein the second dose is delivered at about 24 hours after the first dose.

28. The method of claim 25, further comprising administering to the subject a third dose of EPO delivered within about 20 hours to about 60 hours after the ischemic event.

29. The method of claim 28, wherein the third dose of EPO is delivered within about 8 to 24 hours after the second dose.

30. The method of claim 25, wherein each dose of EPO comprises a subcutaneous, intramuscular, intravenous, or intra-peritoneal injection of EPO.

31. The method of claim 25, wherein each EPO dosage delivered is selected from about 500 IU/kg to about 10000 IU/kg.

32. The method of claim 31, wherein each EPO dosage delivered is selected from about 2500 IU/kg to about 5000 IU/kg.

33. The method of claim 32, wherein each EPO dosage delivered is about 2500 IU/kg.

34. The method of claim 25, wherein the ischemic event is a stroke.

35. The method of claim 34, wherein at least one EPO dosage delivered is about 2500 IU/kg.

36. The method of claim 35, wherein each EPO dosage delivered is about 2500 IU/kg.

37. A method for reducing infarct size in a subject having received an initial exposure to EPO within 6 hours of an ischemic event comprising administering to said subject an amount of EPO between about 1500 IU/kg to about 4500 IU/kg per dose, wherein a first dose of EPO following the initial exposure to EPO is delivered within about 8 to about 26 hours after the initial exposure to EPO followed by a second dose of EPO delivered within about 8 to about 26 hours after the first dose.

38. The method of claim 37, wherein the first dose of EPO is delivered about 24 hours after the ischemic event.

39. The method of claim 38, wherein the second dose is delivered at about 24 hours after the first dose.

40. The method of claim 37, further comprising administering to the subject a third dose of EPO delivered within about 20 hours to about 60 hours after the ischemic event.

41. The method of claim 40, wherein the third dose of EPO is delivered within about 8 to 24 hours after the second dose.

42. The method of claim 37, wherein each dose of EPO comprises a subcutaneous, intramuscular, intravenous, or intra-peritoneal injection of EPO.

43. The method of claim 37, wherein each EPO dosage delivered is selected from about 1800 IU/kg to about 4000 IU/kg.

44. The method of claim 43, wherein each EPO dosage delivered is selected from about 2000 IU/kg to about 3000 IU/kg.

45. The method of claim 44, wherein each EPO dosage delivered is about 2500 IU/kg.

46. The method of claim 37, wherein the ischemic event is a stroke.

47. The method of claim 46, wherein at least one EPO dosage delivered is about 2500 IU/kg.

48. The method of claim 47, wherein each EPO dosage delivered is about 2500 IU/kg.

49. A method for inhibiting apoptosis or inflammation in CNS in a subject after an ischemic event comprising administering to said subject a therapeutically effective amount of EPO, wherein a first dose of EPO is delivered within about 8 to about 26 hours after the ischemic event followed by a second dose of EPO delivered within about 8 to about 26 hours after the first dose.

50. The method of claim 49, wherein the first dose of EPO is delivered about 24 hours after the ischemic event.

51. The method of claim 50, wherein the second dose is delivered at about 24 hours after the first dose.

52. The method of claim 51, further comprising administering to the subject a third dose of EPO delivered within about 20 hours to about 60 hours after the ischemic event.

53. The method of claim 52, wherein the third dose of EPO is delivered within about 8 to 24 hours after the second dose.

54. The method of claim 49, wherein each dose of EPO comprises a subcutaneous, intramuscular, intravenous, or intra-peritoneal injection of EPO.

55. The method of claim 49, wherein each EPO dosage delivered is about 2500 IU/kg.

56. The method of claim 49, wherein the ischemic event is a stroke.

57. The method of claim 56, wherein at least one EPO dosage delivered is about 2500 IU/kg.

58. The method of claim 57, wherein each EPO dosage delivered is about 2500 IU/kg.
